(19) United States
(12) Patent Application Publication
(10) Pub. No.: US 2008/0195061 A1
(43) Pub. Date: Aug. 14, 2008

(54) Method and system for an automated interactive rehydration and calculator thereof

(76) Inventor: Aloyslus N. Fobi, Portland, OR (US)

Correspondence Address: Aloysius N. Fobi, 15541 SE Ogden Dr., Portland, OR, 97236, US

(21) Appl. No.: 11/705,479

(22) Filed: Feb. 12, 2007

(51) Int. Cl. A61M 5/00

(52) U.S. Cl. 604/246

(57) ABSTRACT

A method for re-hydration of an individual is provided. The method obtains biometric information of the individual and inputs it into a processing means. An algorithm determines an amount of fluid to be injested by the individual and a time period using the biometric information. A value of the amount of fluid is displayed. After receiving an indication of fluid being ingested by the individual, the process waits for the time period. Then, an additional amount of fluid to be ingested by the individual is determined. A value of the additional amount of fluid is displayed. The process returns to the determining an amount of fluid after receiving an indication of a diarrhea event. The process pauses for a period of time after receiving an indication of a vomiting event and returns to the determining an amount of fluid.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office, patent file or records, but otherwise reserves all copyright rights whatsoever.

### FIELD OF THE INVENTION

The present invention relates generally to the treatment of dehydration. More particularly, the invention relates to an automated, interactive re-hydration process that enables individuals to calculate the correct amount of fluids for re-hydration.

### BACKGROUND OF THE INVENTION

Worldwide dehydration is responsible for many deaths and millions of visits to medical care providers annually. In the past individuals who were not medically trained frequently gave or took incorrect amounts of fluids when trying to rehydrate during a gastro-intestinal illness or an episode of dehydration. As a result many of these individuals with illnesses or feedings of dehydration present themselves to physicians or hospitals for re-hydration at an annual cost of billions of dollars not including the human cost.

Historically, medicine has sought to treat re-hydration with intravenous fluids, anti-nausea agents, and anti-diarrheal agents. This type of treatment has several barriers, the least of which is cost and access to a limited resource, which is the medical care provider. However, recent literature has shown that optimal re-hydration may be achieved without the need for intravenous fluids or anti-nausea medication. This method involves oral fluid re-hydration. The world health organization and UNICEF have taken an aggressive role towards teaching oral re-hydration in developing countries. These organizations have found that, when able to teach individuals the keys to adequate hydration, the average person is able to avoid dehydration. However, there is quite a bit education that needs to be given to a population in order for this method to work. As such, this education barrier is a significant hindrance to the beneficial spread of the oral re-hydration process and technique.

Modern medicine consistently trains physicians in different methods of providing hydration to their patients. The majority of these methods involve weight-based methods to determine the amount of fluids one would need to administer to their patients. In addition, several medical journal articles have documented the efficacy of orally re-hydrating ill adults and children. As such, this process has become increasingly popular among physicians over intravenous re-hydration.

Historically, others have attempted to address the challenge of treating dehydrated individuals by several methods. One known method is intravenous fluids, which has existed for a very long time but does have several limitations including, without limitation, pain, cost, increased time, risk of infection, and limited ability to employ the process at home. Another method is nasogastric fluid administration, which involves the administration of fluids through a tube passed from the nose into the stomach. This method also has multiple limitations including, but not limited to, pain, cost, increased time, and limited ability to employ in a non-hospital setting.

Those who have attempted to tackle the problem of dehydration in adults by administering fluid by mouth have taken the path of establishing rules of thumb in educating their patients about how much fluid and how frequently their patients should drink a certain amount of fluid. This process usually requires a knowledgeable physician taking the time to educate and determine how much fluid should be obtained and then writing it down for the patient or parent or waiting while the parent or patient writes down the information.

Another well-documented fact is that individuals who are afflicted with a gastro-intestinal disease are susceptible to vomiting. This vulnerability to vomiting increases with the attempt to re-hydrate by drinking fluids aggressively. This places the individual in a position where if the ill individual does not drink fluids, the individual will become dehydrated, and if the individual drinks fluids normally or as fast as their thirst dictates, the individual will most likely vomit. The answer to this problem is smaller but more frequently fluid ingestions. Physiologically this allows the body time to absorb the fluid. As such, even if the individual feels nauseated, he is unlikely to vomit as his body has already absorbed the recently ingested liquid.

Modern Medicine has discussed oral re-hydration in children in several journal articles. There are, however, no published articles that describe an automated process. Traditionally, it requires manual calculation in addition to an instruction to the individual on the amount of fluid to be ingested either in person or in writing. In addition, if the individual is vomiting or has had a diarrheal episode, there is no current method by which to alter the amount of fluid needed without starting the process over and manually adjusting the values and communicating this process again to the individual.

In view of the foregoing, there is a need for an automated method for average individuals to maintain adequate hydration status for themselves and their family without the need of a medical practitioner or the need to overcome a learning curve concerning how to administer the re-hydration fluids.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 is a flow chart of an exemplary process of orally re-hydrating an individual, in accordance with an embodiment of the present invention;

FIG. 2 illustrates schematic diagrams of top, front and side views of an exemplary physical self-contained calculator that implements an oral re-hydration process, in accordance with an embodiment of the present invention; and

FIG. 3 illustrates a typical computer system that, when appropriately configured or designed, can serve as a computer system in which the invention may be embodied.

Unless otherwise indicated, illustrations in the drawings are not necessarily drawn to scale.

### SUMMARY OF THE INVENTION

To achieve the forgoing and other objects and in accordance with the purpose of the invention, a method, system, device and computer program product for automated interactive re-hydration is presented.

In one embodiment, a method for re-hydration of an individual is provided. The method comprises the steps of obtaining biometric information of the individual, inputting the biometric information into a processing means, providing an algorithm for the processing means, determining an amount of fluid to be ingested by the individual and a time period using the biometric information and the algorithm, indicating a value of the amount of fluid, receiving an indication of fluid being ingested by the individual, waiting for the time period after receiving the indication, determining an additional amount of fluid to be ingested by the individual at the end of the time period, outputting a value of the additional amount of fluid, and returning to the indicating. In another embodiment, the method further comprises the step of returning to the determining an amount of fluid after receiving an indication of a diarrhea event after the outputting a value. In another embodiment, the method further comprises the steps of pausing for a period of time after receiving an indication of a vomiting event after the outputting a value and returning to the determining an amount of fluid. In another embodiment, the steps of indicating a value and outputting a value further comprise sending an alert to a user. In another embodiment, the step of determining an amount of fluid further comprises determining a number of hydration steps to complete. In yet another embodiment the step of determining an additional amount of fluid further comprises varying the time period according to a number of hydration steps completed.

In another embodiment, a calculator device for implementing a re-hydration process for an individual is provided. The device comprises a housing, a display mounted on the housing for displaying instructions for a user and the users inputs, an input means mounted on the housing for allowing the user to input biometric information, and a processor means contain in the housing, operating the display and accepting inputs from the input means for processing an algorithm for re-hydration of the individual where the algorithm provides instructions for ingesting fluids at determined times. In another embodiment, the device further includes an event input means for inputting events related to the individual being re-hydrated allowing the algorithm to adjust the instructions and the times. In yet another embodiment, the device further includes a means for alerting the user of instructions for ingesting fluids.

In another embodiment a system for re-hydration of an individual is provided. The system comprises a means for obtaining biometric information of the individual, a means for processing the biometric information to provide a re-hydration schedule, and a means for alerting a user of the schedule. In another embodiment, the system further includes a means for accepting user inputs of events related to the individual being re-hydrated and adjusting the schedule according to the events.

In another embodiment, a computer program product residing on or being distributed across one or more computer readable mediums having a plurality of instructions stored thereon is provided. The computer program product which, when executed by one or more associated processors, cause the one or more processors to obtain biometric information of the individual, process the biometric information to determine an amount of fluid to be ingested by the individual and a time period using an algorithm, present to a user a value of the amount of fluid, receive an indication of fluid being ingested by the individual, wait for the time period after receiving the indication, determine an additional amount of fluid to be ingested by the individual at the end of the time period, display to the user a value of the additional amount of fluid, and return to present to a user a value of the amount of fluid. In another embodiment, the computer program product upon receiving an indication of a diarrhea event from the user returns to process the biometric information. In still another embodiment, the computer program product upon receiving an indication of a vomiting event from the user returns to process the biometric information. In another embodiment, the computer program product sends an alert to the user when the amount or additional amount of fluid is presented or displayed. In another embodiment, a number of hydration steps to complete is determined. In yet another embodiment, the time period is varied according to a number of hydration steps completed.

Other features, advantages, and object of the present invention will become more apparent and be more readily understood from the following detailed description, which should be read in conjunction with the accompanying drawings.

### DETAILED DESCRIPTION OF THE PREFERRED EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognized a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternatives embodiments do not necessarily imply that the two are mutually exclusive.

The present invention will now be described in detail with reference to embodiments thereof as illustrated in the accompanying drawings.

The terms biometric information, input, and fluids are frequently used in this disclosure and are defined as follows. Biometric information is any information that reflects a measurement of the human body or physiology. These include but are not limited to age, weight, sex, height, body surface area, gross and net fluid movement, body mass index (BMI), temperature, etc. Input is any method of adding information for an automated machine or computer to process, for example, without limitation, typing, voice input, etc. Fluid is used herein to describe the substance to be ingested by the user and may be any chemical that an individual might ingest in order to maintain a normal balance of fluid and or electrolytes in the body. These substances may be delivered in a solid form such as, but not limited to, ice or popsicles or some other semi-solid state, for example, without limitation, a gelatin.

Preferred embodiments of the present invention involve processes whereby individual patient information, for example, without limitation, biometric information, is entered into an automated system. This automated system calculates how much of a given fluid should be given to that individual at a specified schedule with or without the capability to notify the individual of when to give the desired amount of fluid. The individual can be alerted as to how much fluid to ingest by numerous different methods including but not limited to digital read out, print outs, e-mail, fax, text, website, phone call, voice command, etc.

Embodiments of the present invention may be used by practitioners who do not have the time necessary to educate their patients in oral re-hydration. Some embodiments may also be used by parents who are taking care of ill children at risk for dehydration. Individuals and organizations in developing countries will also find embodiments of the present invention useful, as these individuals and organizations can leverage their time by distributing re-hydration devices according to these embodiments. Without the need to see a physician or be educated about the re-hydration process, the individual is able to immediately begin the re-hydration treatment using an embodiment of the present invention thereby increasing the speed of re-hydration.

One specific embodiment of the present invention is a physical re-hydration calculator with an alarm that contains several digital input keys that enable the user to interface with the re-hydration calculator and input biometric information such as, but not limited to, age, weight, height, BMI, etc. The input information is used to calculate a specific biometrically based re-hydration schedule from the user's unique biometric profile. From that information the calculator, at different intervals, alerts the user how much fluid to give. The user may be notified by a combination of audio, visual, and tactile alert systems when it is time to take another dose of fluid. Once the fluid is ingested, the user inputs that information into the calculator and the timer counts down until it is time to take the next dose. Included within this re-hydration calculator is, without limitation, an optional algorithm that enables the user to enter that they had a diarrheal bowl movement or a vomiting episode. Once the user enters that they had a diarrheal bowel movement or a vomiting episode, the algorithm automatically adjusts how much fluid the user should take during the next intervals.

The preferred embodiment of the present invention is separate and distinct from the numerous medical dosing calculators that exist for medical practitioners that enable them to properly prescribe a certain dose of medication. Among many of the distinctions, without limitation, is the interactive nature of the present device with the end user or patient. For example, without limitation, every answer that the user gives as time goes on may affect the result of the next value that is output. This provides the user with a personalized re-hydration process based on their individual biometric information. Other distinctions are alarm and notification abilities that are integrally linked to the user's biometric information such that if one data point is changed, it may or may not affect the remaining data. The preferred embodiment of the present invention is also distinct from any of the intravenous pumps that exist that enable a user to input an infusion rate of any given substance as those devices are for use with intravenous and direct enteral (tubes that directly feed into the bowel). The preferred embodiment is also intended for use by the layperson in addition to the practicing medical professional. The preferred embodiment describes an automated process that uses algorithms that are calculated by computer processors, which in turn leads to an intuitive and easy to use interface, which accomplishes the desired effect.

The preferred embodiment entails but is not limited to several components such as, but not limited to, knowledge of the intended user's biometric information, a user interface, an algorithm that calculates the amount of fluid and the time to be given, an output device, and a timer with notification or alarm capabilities.

It has long been established that the best method to assess how much fluid a given individual needs to maintain adequate hydration at baseline is to use information that is specific as possible to that individual. The basic premise is that the amount of fluid contained in the body of each person is proportional to the size of their body. There are several measurements of biometrics that are frequently used to assess how much fluid a person may need as a function of time. The most commonly used in medicine is weight. Other variables that may be included for a more accurate determination of fluid needs are, without limitation, height, age, body surface area, volume, and BMI.

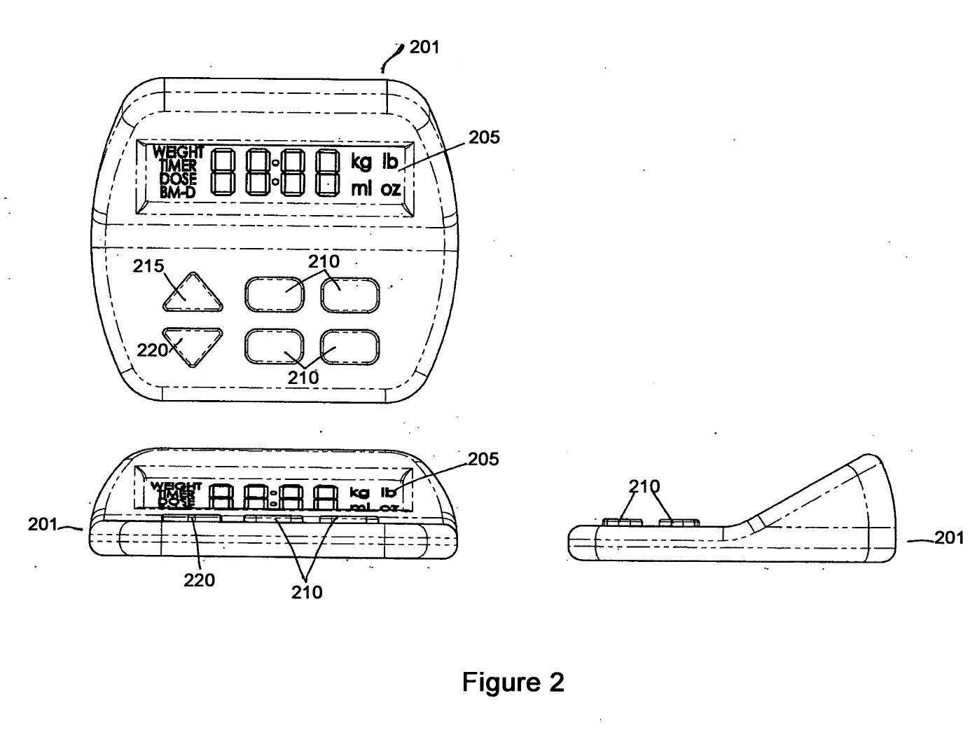

In the preferred embodiment, the user interface may comprise, without limitation, any device into which a user can enter their individual biometric information. This may take place for example, without limitation, on a key pad of a physical calculator, entry over the internet, or through a mobile or cellular device. Specifically, on an embodiment comprising a physical calculator as demonstrated by way of example in FIG. 2, the user interface comprises a screen to display information such as, but not limited to, the amount of fluid to ingest and buttons for the user to input information such as, but not limited to, biometric information. On a web-based embodiment, the user has the option to choose from the appropriate weight or other biometric information on a drop down box. Also, in some web-based embodiments, a menu may have the capability to choose between units of measurement if necessary such as, but not limited to, pounds or kilograms. In the preferred embodiment, the user interface also has a method for the user to indicate if the user has had a diarrheal bowel movement or a vomiting episode. For example, without limitation, in a physical calculator, if the user has had an episode of emesis or vomiting the user would press a vomiting button. A similar process takes place with loose stools triggering the need to press a diarrhea button.

The preferred embodiment comprises a re-hydration algorithm that utilizes and calculates appropriate fluid amounts based on input individual biometric information. In the preferred embodiment of the algorithm the biometric information to be entered is the user's weight. The biometric data is multiplied by a coefficient that is known to be an accurate reflection of fluid needs based on the average needs of an individual with that biometric information. In the case of ongoing fluid losses for example, without limitation, diarrhea or vomiting, the user presses the button that indicates that the user has had a diarrheal stool or a vomiting episode. In the event of fluid loss through a diarrheal stool, the algorithm increases the overall amount of fluid needed based upon the average fluid loss for a diarrheal stool for individuals with that biometric information. If the user indicates that they had ongoing net fluid loss through vomiting by pressing the vomiting button, the algorithm takes that into account. In this case a vomiting episode triggers a pause and restarts the user to the beginning of the re-hydration process.

The amount of fluid to be given is separated by a fixed or variable time interval dependent on the needs of the user. This time period is preprogrammed into the algorithm such that the user need not know when or how much fluid to ingest. As the user successfully is able to drink and tolerate increasing amounts of fluid the algorithm increases the amount of fluid and decreases the frequency that the user needs the fluid until the user is well.

In order for the user to know how much fluid they should be drinking, the user needs to be alerted of this information at the appropriate time. This can be accomplished through several methods including but not limited to an alarm with audio, visual, or tactile cues. In an embodiment implementing a physical calculator, the physical calculator may also use a display such as, but not limited to, an LED or LCD display to prompt entries from the keyboard and to provide visual conformation that the desired entry data is correct. For embodiments implementing a computer or web-based program, a computer screen “pop up” screen with notification of how much fluid to give or an e-mail or text message may be used to alert the user. Some users may wish to view their schedule in the form of a print out, which is possible in some embodiments; however, these embodiments do not take into account any of the dynamic and interactive factors of the preferred embodiment for example, without limitation, taking into account a diarrheal bowel movement or a vomiting episode.

After the user has entered the biometric information and initiated the start program in the preferred embodiment, the algorithm issues a command of how much fluid to ingest. Once that amount is given, the user indicates that the fluid has been given, at which point a timer counts down until the timer reaches a specified time, specific to the user's biometric information. Once the timer has counted down to that time, a notification tool such as, but not limited to, an alarm is activated reminding the user to ingest more fluid along with the amount of fluid to be ingested. This pattern continues until the user reaches the end of the algorithm.

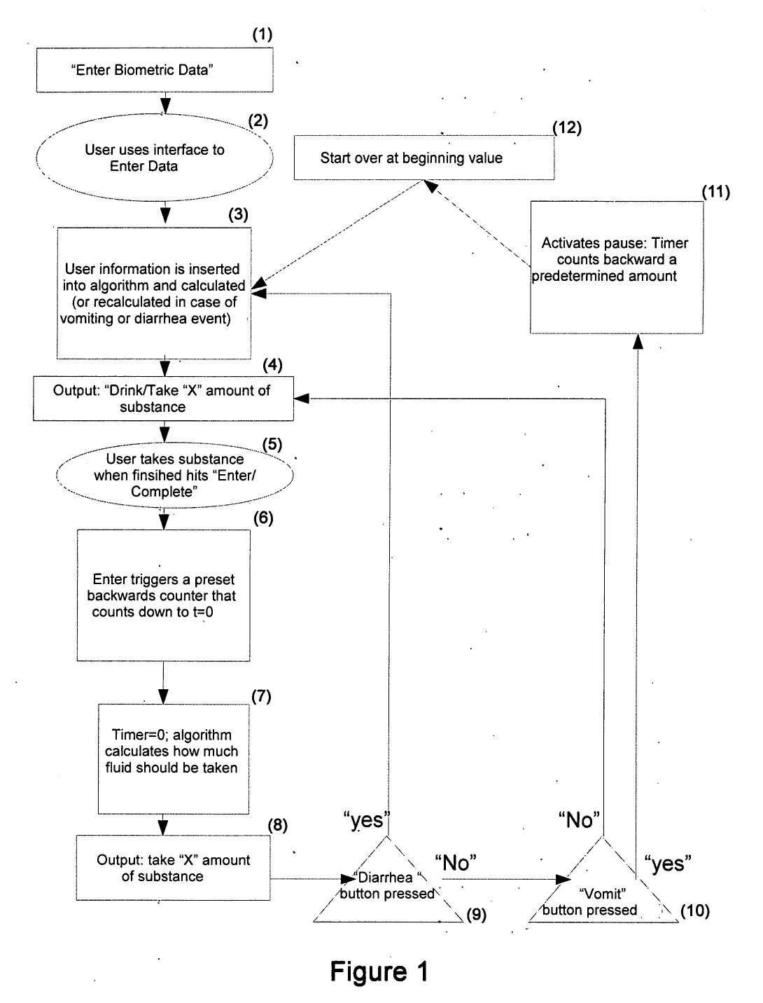

FIG. 1 is a flow chart of an exemplary process of orally re-hydrating an individual, in accordance with an embodiment of the present invention. In step 1, an interface of a calculation device requests certain biometric information from a user. The user enters the required biometric data into the interface of the device in step 2. In step 3, this information is processed using an algorithm to determine how much fluid is needed and when it should be ingested by the user. An exemplary algorithm is described above by way of example with respect to the preferred embodiment. In step 4 of the present embodiment, the interface outputs instructions for the user to ingest a certain amount of fluid. In some embodiments, this output may be accompanied by an alert to the user such as, but not limited to an alarm or a flashing light.

Once the user ingests the fluid, the user indicates this on the interface in step 5 for example, without limitation by pressing an enter key or a complete button, and the algorithm proceeds onto the next dose at the next time. After the fluid is ingested a timer counts down to the next ingestion event through a preset timer that counts down to time t=0 in step 6. The amount of time on the preset timer may vary by user according to the user's biometric information and also varies throughout the process, generally allowing for more time between fluid ingestion as the process progresses. Once t=0 the algorithm calculates how much fluid should be ingested in the next dosage in step 7. In step 8 the calculation device indicates how much fluid is to be taken by the user by displaying this information of the user interface. As in step 4, this display may be accompanied by an alert.

Step 9 is an optional step where the algorithm determines if a diarrhea button has been pressed. If the diarrhea button has been pressed, the normal process is bypassed and the algorithm returns to step 3 to recalculate total amount of fluid to be given with this new information being taken into account. If the diarrhea button has not been pressed, the algorithm goes to step 10 to determine if a vomit button has been pressed. If the vomit button has not been pressed, the algorithm assumes that no vomiting or diarrheal events have occurred and the algorithm returns to step 4 to indicate to the user the amount of fluid to take. Optionally, in some alternate embodiments, if the vomit button has been pressed, the algorithm goes to step 11 and activates a timer for a predetermined amount of time. Once the vomit button has been activated, the timer counts backwards from a preset value until the timer reaches t=0. Then, the algorithm proceeds to step 12. In step 12 the user starts at the beginning dosage amount that was originally calculated in step 3. The user does not need to re-enter their biometric information at this step. The algorithm then proceeds to step 3 to recalculate the amount of fluid that the user should take. This cycle continues potentially indefinitely until the user is no longer ill or feels adequately hydrated. It is possible for a preset amount of hydration steps to be inputed into the algorithm. A typical end point is 60 minutes in some practical applications.

FIG. 2 illustrates schematic diagrams of top, front and side views of an exemplary physical self-contained calculator 201 that implements an oral re-hydration process, in accordance with an embodiment of the present invention. In the present embodiment, calculator 201 is a portable, self-contained device with the capability of processing and outputting information as well as alerting the user of the presence of output data for example, without limitation, an alarm or a voice command. In the present embodiment, calculator 201 is a palm-sized device that is operated by a small traditional watch battery or existing solar (photovoltaic device) technology. Calculator 201 comprises a screen 205 and multiple buttons 210. Buttons 210 may vary in function depending on the application of calculator 201. Some examples of buttons include without limitation, an enter key, a diarrhea button, a vomiting button, an on/off button, and a unit change button. The present embodiment shows four buttons 210 by way of example; however, alternate embodiments may have more or less buttons with various functions included on calculator 201.

In the present embodiment, when the user presses the on/off button, calculator 201 is activated and requests biometric information such as, but not limited to, weight, height, etc. Screen 205 enables the user to see the biometric information as he enters the data. Screen 205 may be an LED or LCD display. Alternate embodiments may comprise various other types of displays such as, but not limited to a digital display. In the present embodiment, calculator 201 has two arrows buttons, an up button 215 and a down button 220. To enter biometric information into calculator 201, the user toggles up or down with up button 215 and down button 220 until the appropriate biometric information is being shown on screen 205, at which point the user presses the enter key to indicate that the biometric information is correct. In alternate embodiments, the calculator may have a keypad that the user may use to type in their biometric information.

After the user enters their biometric information, a processor in calculator 201 displays how much fluid the individual should receive on screen 205. The user ingests this amount and then notifies calculator 201 that the user has taken the fluid by pressing a button such as, but not limited to the enter button, a complete button, or a fluid ingested button. A timer in calculator 201 counts down until it is time to ingest the next amount of fluid. The user is alerted then notified that it is time to ingest the next amount of fluid by calculator 201. This notification may be accomplished through many means such as, but not limited to, an alarm, a voice command from a speaker in calculator 201, a flashing light, vibration, tactile stimulation, or flashing screen, linked to a peripheral device such as a mobile phone, pager, SMS messaging, etc.

The present embodiment may also incorporate means to enter other data such as, but not limited to, a diarrheal or vomiting event for example, without limitation, a vomiting or a diarrhea button. If the user presses the vomiting or diarrhea button, an algorithm in calculator 201 calculates a new amount for fluid intake and continues with the timer, alerting the user when the next fluid dosage is to be taken. The present embodiment may also comprise a unit change button that enables the user to change the input and/or output units for calculator 201. For example, without limitation, the unit change button may enable the user to change the unit of weight between pounds and kilograms, the unit of height between inches and centimeters, and the unit of fluid measurement between ounces and milliliters.

In an alternate embodiment of the present invention, a website or software that could be used by personal computers or individuals with internet access may incorporate an algorithm for oral re-hydration. In a Web or computer based calculator, the actual algorithm may be stored on a central computer or server or may be downloaded from a location or disc to a personal computer. The functionality and the method of use are very similar with regards to the goals being met and many of the problems solved by the physical calculator embodiment shown by way of example in FIG. 2.

The web-based embodiment of this invention has the potential benefit of the user being able to access it anywhere there is internet service and may be accessed by multiple people at the same time. The web-based embodiment of the invention takes the form of an interactive website. On this website a user is prompted to enter their biometric information. This may be done for example, without limitation, by using drop-down boxes or typing the information into the computer. Upon doing so the website activates the algorithm and starts the re-hydration process as shown by way of example in FIG. 1. The present embodiment may also comprise a menu that can be used for various functions such as, but not limited to, changing the input and output units and indicating that the user has experienced a diarrheal or vomiting episode. The website and the algorithm also have the ability to be linked to several different output media separately or jointly. As such, the invention can notify the user to take a certain amount of fluid by e-mail or other means such as, but not limited to, a pop-up notification or an audio alert. Another benefit of the web-based embodiment is the case with which multimedia demonstration of the process can be displayed. This enables the program to demonstrate to the user exactly when and how to hydrate himself or another individual. A software based embodiment functions the same as a web based embodiment. However, the user requires access to a computer on which the re-hydration software has been installed.

The description above describes the user as the individual that is being re-hydrated. However, there may be cases in which the user of the re-hydration calculator may be someone other than the dehydrated individual for example, without limitation, if a parent is taking care of a sick child. Also, embodiments of the present invention may be used by health care providers to re-hydrate their patients.

Embodiments of the present invention are not intended to replace the judgment of a trained professional in assessing the best course of treatment for an ill individual. Instead, these embodiments may be used to decrease the rate of progression to serious illness and dehydration. If the individual already appears ill, the individual may still benefit from these embodiments; however individuals that are ill may still need the attention of a licensed medical professional.

Those skilled in the art will readily recognize, in accordance with the teachings of the present invention, that any of the foregoing steps and/or system modules may be suitably replaced, reordered, removed and additional steps and/or system modules may be inserted depending upon the needs of the particular application, and that the systems of the forgoing embodiments may be implemented using any of a wide variety of suitable processes and system modules, and is not limited to any particular computer hardware, software, middleware, firmware, microcode and the like.

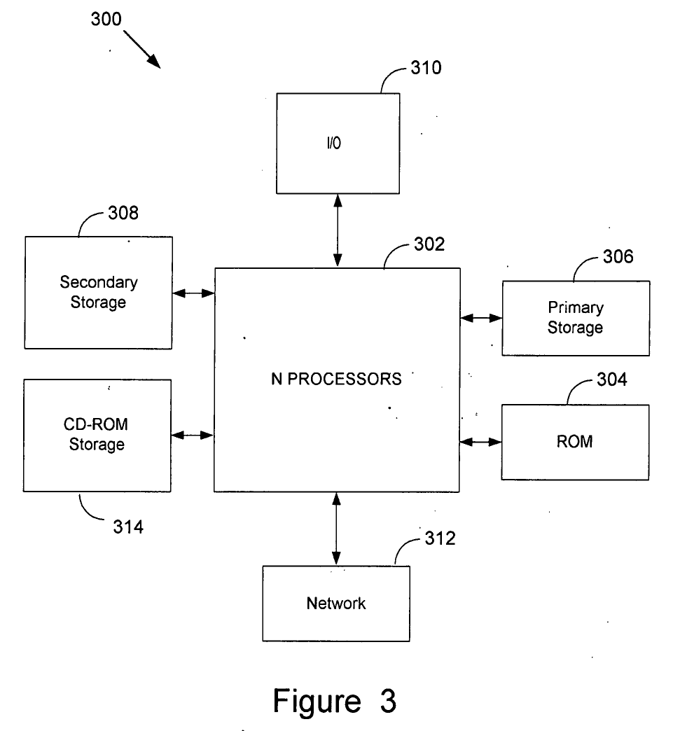

FIG. 3 illustrates a typical computer system that, when appropriately configured or designed, can serve as a computer system in which the invention may be embodied. The computer system 300 includes any number of processors 302 (also referred to as central processing units, or CPUs) that are coupled to storage devices including primary storage 306 (typically a random access memory, or RAM), primary storage 304 (typically a read only memory, or ROM). CPU 302 may be of various types including microcontrollers (e.g., with embedded RAM/ROM) and microprocessors such as programmable devices (e.g., RISC or SISC based, or CPLDs and FPGAs) and unprogrammable devices such as gate array ASICs or general purpose microprocessors. As is well known in the art, primary storage 304 acts to transfer data and instructions uni-directionally to the CPU and primary storage 306 is used typically to transfer data and instructions in a bi-directional manner. Both of these primary storage devices may include any suitable computer-readable media such as those described above. A mass storage device 308 may also be coupled bi-directionally to CPU 302 and provides additional data storage capacity and may include any of the computer-readable media described above. Mass storage device 308 may be used to store programs, data and the like and is typically a secondary storage medium such as a hard disk. It will be appreciated that the information retained within the mass storage device 308, may, in appropriate cases, be incorporated in standard fashion as part of primary storage 306 as virtual memory. A specific mass storage device such as a CD-ROM 314 may also pass data uni-directionally to the CPU.

CPU 302 may also be coupled to an interface 310 that connects to one or more input/output devices such as such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 302 optionally may be coupled to an external device such as a database or a computer or telecommunications or internet network using an external connection as shown generally at 312, which may be implemented as a hardwired or wireless communications link using suitable conventional technologies. With such a connection, it is contemplated that the CPU might receive information from the network, or might output information to the network in the course of performing the method steps described in the teachings of the present invention.

It will be further apparent to those skilled in the art that at least a portion of the novel method steps and/or system components of the present invention may be practiced and/or located in location(s) possibly outside the jurisdiction of the United States of America (USA), whereby it will be accordingly readily recognized that at least a subset of the novel method steps and/or system components in the foregoing embodiments must be practiced within the jurisdiction of the USA for the benefit of an entity therein or to achieve an object of the present invention. Thus, some alternate embodiments of the present invention may be configured to comprise a smaller subset of the foregoing novel means for and/or steps described that the applications designer will selectively decide, depending upon the practical considerations of the particular implementation, to carry out and/or locate within the jurisdiction of the USA. For any claims construction of the following claims that are construed under 35 USC §112(6) it is intended that the corresponding means for and/or steps for carrying out the claimed function also include those embodiments, and equivalents, as contemplated above that implement at least some novel aspects and objects of the present invention in the jurisdiction of the USA. For example, the actual act of physically inputting the data by the user and the display of the rehydration instructions to the user from the graphical user interface may be performed and/or located outside of the jurisdiction of the USA while the remaining method steps and/or system components of the forgoing embodiments are typically required to be located/performed in the US for practical considerations.

Having fully described at least one embodiment of the present invention, other equivalent or alternative means for implementing a re-hydration method according to the present invention will be apparent to those skilled in the art. For example, without limitation, a re-hydration calculator may be incorporated into a wristwatch or a cellular phone, making the device very portable. The invention has been described above by way of illustration, and the specific embodiments disclosed are not intended to limit the invention to the particular forms disclosed. The invention is thus to cover all modification, equivalents, and alternatives falling within the spirit and scope of the following claims.

What is claimed is:

1. A method for re-hydration of an individual, the method comprising the steps of:
obtaining biometric information of the individual;
inputting said biometric information into a processing means;
providing an algorithm for said processing means;
determining an amount of fluid to be ingested by the individual and a time period using said biometric information and said algorithm;
indicating a value of said amount of fluid;
receiving an indication of fluid being ingested by the individual;
waiting for said time period after receiving said indication; and
determining an additional amount of fluid to be ingested by the individual at the end of said time period.

2. The method as recited in claim 1, further comprising the step of outputting a value of said additional amount of fluid.

3. The method as recited in claim 1, further comprising the step of returning to said step of indicating.

4. The method as recited in claim 1, further comprising the step of returning to said determining an amount of fluid after receiving an indication of a diarrhea event after said outputting a value.

5. The method as recited in claim 4, further comprising the steps of:
pausing for a period of time after receiving an indication of a vomiting event after said outputting a value; and
returning to said determining an amount of fluid.

6. The method as recited in claim 5, where said steps of indicating a value and outputting a value further comprise sending an alert to a user.

7. The method a recited in claim 6, where said step of determining an amount of fluid further comprises determining a number of hydration steps to complete.

8. The method as recited in claim 7, where said step of determining an additional amount of fluid further comprises varying said time period according to a number of hydration steps completed.

9. A calculator device for implementing a re-hydration process for an individual, the device comprising:
a housing;
a display mounted on said housing for displaying instructions for a user and said users inputs;
an input means mounted on said housing for allowing said user to input biometric information; and
a processor means contain in said housing, operating said display and accepting inputs from said input means for processing an algorithm for re-hydration of the individual where said algorithm provides instructions for ingesting fluids at determined times.

10. The device as recited in claim 9, further comprising an event input means for inputting events related to the individual being re-hydrated allowing said algorithm to adjust said instructions and said times.

11. The device as recited in claim 10, further comprising a means for alerting said user of instructions for ingesting fluids.

12. A system for re-hydration of an individual, the system comprising:
a means for obtaining biometric information of the individual;
a means for processing said biometric information to provide a re-hydration schedule; and
a means for alerting a user of said schedule.

13. The system as recited in claim 12, further comprising a means for accepting user inputs of events related to the individual being re-hydrated and adjusting said schedule according to said events.

14. A computer program product residing on or being distributed across one or more computer readable mediums having a plurality of instructions stored thereon which, when executed by one or more associated processors, cause the one or more processors to:
obtain biometric information of the individual;
process said biometric information to determine an amount of fluid to be ingested by the individual and a time period using an algorithm;
present to a user a value of said amount of fluid;
receive an indication of fluid being ingested by the individual;
wait for said time period after receiving said indication;
determine an additional amount of fluid to be ingested by the individual at the end of said time period;

15. The computer program product as recited in claim 14, in which upon receiving an indication of a diarrhea event from said user returns to process said biometric information.

16. The computer program product as recited in claim 15, in which upon receiving an indication of a vomiting event from said user returns to process said biometric information.

17. The computer program product as recited in claim 16, in which an alert is sent to said user when said amount or additional amount of fluid is presented or displayed.

18. The computer program product as recited in claim 17, in which a number of hydration steps to complete is determined.

19. The computer program product as recited in claim 18, in which said time period is varied according to a number of hydration steps completed.

20. The computer program product as recited in claim 14, in which the one or more processors is further caused to display to said user a value of said additional amount of fluid.

21. The computer program product as recited in claim 14, in which the one or more processors is further caused to return to presenting to a user a value of said amount of fluid.

22. A method for re-hydration of an individual comprising:
Steps for obtaining biometric information of the individual;
Steps for processing said biometric information to provide a re-hydration schedule; and
Steps of alerting a user of said schedule.
